(19) United States
(12) Patent Application Publication
(10) Pub. No.: US 2009/0222069 A1
(43) Pub. Date: Sep. 3, 2009

(54) LIGHT MODULATION OF CELL FUNCTION

(75) Inventors: Steffen Bjorn Petersen, Aalborg (DK); Maria Teresa Neves-Petersen, Aalborg (DK); Olaf-Georg Issinger, Aarslev (DK); Birgitte B. Olsen, Odense (DK); Torben Snabe, Hadsten (DK); Soren Klitgaard, Aalborg (DK)

Correspondence Address: DARBY & DARBY P.C., P.O. BOX 770, Church Street Station, New York, NY, 10008-0770, US

(73) Assignee: Aalborg Universitet, Aalborg (DK)

(21) Appl. No.: 12/093,918

(22) PCT Filed: Nov. 16, 2006
(86) PCT No.: PCT/DK2006/000637

§ 371 Date: Jan. 12, 2009

Related U.S. Application Data
(60) Provisional application No. 60/737,505, filed on Nov. 16, 2005.

(30) Foreign Application Priority Data
Nov. 16, 2005 (DK) PA 2005 01603

(51) Int. Cl. A61N 5/067; C12N 13/00; A61N 5/06

(52) U.S. Cl. 607/89; 435/173.1; 435/173.7; 607/88; 604/20

(57) ABSTRACT

The present invention relates to the field of light induced therapy. The invention relates more particularly to a method of modulating receptor function of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to modulate said receptor function.

### TECHNICAL FIELD

The present invention relates to the field of light induced therapy. The invention also relates to a method of treatment of cells such as cancer cells in a subject by illumination with light, and to an apparatus for performing said therapy. The invention also relates to a method of modulating receptor function of cells having receptor proteins using light.

### BACKGROUND OF THE INVENTION

Natural UV irradiation is the major cause of more than 1 million cases of non-melanoma skin cancer arising each year. It causes DNA damage and epigenetic effects in response to DNA damage. Skin responds to UV light by activating numerous signal transduction pathways, such as the mitogen-activated protein kinase signaling cascades that coordinate cell cycle arrest, up-regulation of DNA damage repair pathways, and apoptosis. Whereas extra cellular signal-regulated kinases (ERKs) are critical in response to mitogenic stimuli under normal conditions, p38 kinase and c-Jun NH2-terminal kinases (JNKs) are activated in response to stress, such as exposure to UV irradiation. The epidermal growth factor receptor (EGFR), an activator of mitogen-activated protein kinases (MAPK) signaling pathway, is rapidly activated following exposure to UV light. EGFR activation following UV exposure is a result of both the induction of EGFR ligands and the inactivation of phosphatases that would otherwise inactivate the receptor tyrosine kinase. UV-induced activation of EGFR up-regulates several MAPK and PI3K/AKT signaling pathways that control epidermal cell division and cell death. AKT plays a central role in mediating critical cellular responses including cell growth and survival, angiogenesis, and transcriptional regulation. This protein kinase is activated by insulin and various growth and survival factors and functions in a wortmannin-sensitive pathway involving PI3 kinase. AKT promotes cell survival by inhibiting apoptosis by means of its ability to phosphorylate and inactivate several targets.

Phototherapy is a well-known therapy form. UV light is in general subdivided into three regions, an UVA, UVB and UVC region, where UVA is from 400 nm-315 nm, UVB is from 315 nm-280 nm and UVC 280 nm-100 nm. Narrow band UVB phototherapy is used as a treatment for skin eruptions. It has been a standard therapy in hospitals and clinics since its invention at the Mayo Clinic in the 1920s. Narrow band UVB is the treatment of choice for people with moderately to severe psoriasis (covering 20 percent or more of their body) who have not responded to topical ointments. Narrow band UVB is also used for severe cases of eczema and itching from any cause. Narrow band UVB improves skin diseases because the immune cells of the skin, overactive in many skin diseases, are inactivated by UVB at particular fluency values. Excessive exposure causes premature aging of the skin and increases the risk of skin cancer.

Broad-band UVB refers to treatment with a range of wavelengths between 280 nm and 315 nm. Narrow-band UVB refers e.g. to a specific wavelength of 311 to 312 nm. This range has proved to be the most beneficial component of natural sunlight for treatment of psoriasis and looks promising in the treatment of some other skin conditions including atopic eczema and vitiligo, pruritus, lichen planus, polymorphous light eruption and early cutaneous T-cell lymphoma.

U.S. Pat. No. 4,558,700 relates to a UV radiation device for phototherapy of dermatoses, especially psoriasis, which device produces UV radiation, the radiation intensity E₂ of which present in the effective area in the wavelength range below 300 nm is substantially less than the radiation intensity E₁ in the wavelength range between 300 and 310 nm, the radiation dose being between 0.7 and 1.0 times the erythema threshold dose. The limitation of the radiation intensity below 300 is described as significant from the point of view that this radiation below 300 nm in combination with radiation in the wavelengths between 300 and 310 nm can lead to a reversal of the therapeutic effect.

DE patent application 102 33 839 relates to a UVA irradiation device, which has source(s) emitting radiation of wavelength over 340 nm, with at least a fraction in the 340-400 nm range, onto the surface to be treated, and has means of generating pulses on the surface to be treated, such that each pulse has an energy density of 0.05-50 J/cm2 on this surface with a peak intensity of over 0.5 W/cm2 and under 100 kW/cm2. The device is used for phototherapy, which is useful for treating skin diseases, e.g. atopical eczema, cancers, inflammation and neurodermatitis.

PUVA is a type of ultraviolet radiation treatment used for severe skin diseases. PUVA is a combination treatment which consists of Psoralens (P) and then exposing the skin to long wave ultraviolet radiation (UVA). It has been available in its present form since 1976. Psoralens are compounds found in many plants which make the skin temporarily sensitive to UVA. The ancient Egyptians were the first to use psoralens for the treatment of skin diseases thousands of years ago.

As a treatment modality for malignant and certain non-malignant diseases, photodynamic therapy (PDT) involves a two step protocol which consists of the (selective) uptake and accumulation of a photosensitizing agent in target cells and the subsequent irradiation with light in the visible range. Reactive oxygen species (ROS) produced during this process cause cellular damage and, depending on the treatment dose/severity of damage, lead to either cellular repair/survival, apoptotic cell death or necrosis.

There are known methods that interfere with the function of living cells in patients, such as above mentioned PUVA and PDT. However, little is known about the molecular mechanisms underpinning the therapeutic effects that have been found.

While a non-malignant cell has few receptor proteins on its surface, an over-expression of growth factor receptors are observed on the cellular membrane of many malignant cells. Cell-surface receptors, such as the epidermal growth factor receptor EGFR, are proteins that bind external ligands (activators) e.g. hormones such as EGF, and transmit the external signal inside the cell, ultimately resulting in processes such as cell growth and differentiation. These receptors reside on the cell membrane and typically consist of three parts: a sensing, extra cellular segment; a transmembrane segment; and an intra cellular segment that transmits the signal inside the cell. Abnormal cell signal transduction arising from receptor tyrosine kinases due to mutation or over-expression has been implicated in the initiation and progression of a variety of human cancers.

The epidermal growth factor receptor (EGFR), also known as HER1/Erb-B1 belongs to the ErbB family of receptor tyrosine kinases. Binding of ligands such as EGF and TGF, leads to homo- and heterodimerization of the receptors. Dimerization in the case of EGFR leads to autophosphorylation of specific tyrosine residues in the intracellular tyrosine kinase domain. These phosphorylated tyrosine residues serve as docking sites for other kinases e.g. phosphatidylinositol-3-kinase (PI3K) and adaptor proteins e.g. Shc and Grb2. This in turn initiates cascades of intracellular signaling pathways including the PI3K-AKT pathway and the Ras-Raf-MAPK pathway. Activation of the ErB receptor family triggers a number of different responses including mitogenesis, apoptosis, cellular motility, angiogenesis and differentiation. Activation of the MAPK and PI3K/AKT signaling pathways lead to the expression of mitogenic factors, which can induce the malignant conversion of keratinocytes, tumor progression and invasive potential. This is supported by the finding that the EGF receptor is overexpressed or subject to uncontrolled signaling in a number of solid tumors and is often associated with poor prognosis and advanced disease. The current strategy in targeting the ErbB receptors includes monoclonal antibodies and small-molecule kinase inhibitors, which inhibit autophosphorylation and downstream signaling.

Examples of receptor tyrosine kinases are e.g. EGF, PDGF, IGF-1, NGF and VEGF receptors. Especially the EGF-receptor is highly expressed in a variety of solid tumours, and in many instances it is also expressed in a mutated form.

The EGF receptor has thus been detected in 28 benign skin tumors, in 11 of 15 condylomata acuminata, whereas the investigated mesenchymal tumors and normal skin as a control were receptor negative. 6 of 18 basal cell epitheliomas bound EGF specifically. In the group of precancerous and malingnant skin tumors, 7 of 8 squamous cell carcinomas had the highest number of EGF binding sites, whereas 5 malignant melanomas were receptor negative (Bauknecht et al. (1985) Epidermal growth factor receptors in different skin tumors. Dermatologica, 171, 16-20).

Investigations of 25 patients with vulvar squamous cell carcinoma (VSCC), 10 patients with vulva condyloma acuminate (VCA), 15 patients with vulvar intra-epithelial neoplasm and 5 patients with vulvar normal squamous cells (VNSC) showed the presence of EGFR in various cells. This finding led to the suggestion that the TGF-alpha-EGFR system maintains the growth of normal squamous cells and, in part maintains the growth of dysplastic and neoplastic squamous cells in the vulva. EGF expression was an early sign of neoplasia. The expression of EGFR with the over-expression of its two ligands contributed to the proliferation and dysplastic and neaoplastic squamous cells in VIN II and VCA. Hence EGFR expression appeared to contribute to essential neoplastic abnormalities in 64% of the VSCC (Wu et al. (2001) Med. Electron Microsc. 34, 179-184).

Evidence for EGFR as a target for treatment of lung, head and neck cancers has also been established (Herbst and Langer (2002) EGFRs as a target for cancer treatment: the emerging role of IMC-C225 in the treatment of lung and head and neck cancers. Semin. Oncol. 29, 27-36).

Human papillomas are caused by papilloma virus. A correlation has also been established between increases in EGFR reactivity in the group of mucosal lesions in which viral DNA was more frequently detected than viral antigen, suggesting that viral DNA may play a role in basal cell stimulation (Viac et al. (1987) Virus expression. EGF and transferrin receptor in human papillomas. Virchows Arch A Pathol Anat Histopathol, 411, 73-7).

Presently there are two ways in which EGFR is targeted: (i) by monoclonal antibodies, such as cetuximab and (ii) small molecule tyrosine kinase inhibitors, such as gefitinib and erlotinib. Both approaches have been approved for treatment of colon and lung cancer. Inhibitors targeting other molecules, e.g. vascular endothelial cell targeting agents, matrix metalloproteinase inhibitors, farnesyltransferase inhibitors, retinoids, proteosome inhibitors, and raf/MAPkinase (mitogen-activated protein kinase) inhibitors are also under development. Many of these agents, such as bortezomib, have demonstrated promise in the fields of non-small cell lung cancer (NSCLC).

Since the tyrosine kinases receptors are the ultimate upstream signaling molecules it is plausible to assume that inactivation of these primary molecules will consequently lead to inhibition of downstream reactions.

There still exists a need for new therapies in view of the large number of patients suffering from e.g. cancer in which receptor tyrosine kinases are implicated, and the lack of effective treatments.

It is the object of the invention to provide a treatment and a device for phototherapy of cells having receptor proteins such as receptor tyrosine kinases which makes it possible to achieve an effective treatment using short radiation times, low fluency and a limited number of treatments.

### SUMMARY OF THE INVENTION

One aspect of the invention provides a method of inhibiting proliferation of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said proliferation.

Another aspect of the invention provides a method for inducing apoptosis of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to induce said apoptosis.

Another aspect of the invention provides a method of inhibiting growth of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said growth of cells.

Another aspect of the invention provides a method of inhibiting signal transduction into cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said signal transduction into the cell.

Another aspect of the invention provides a method of inhibiting cell signalling of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said cell signalling.

Another aspect of the invention provides a method of inhibiting cellular receptor activation of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said cellular receptor activation.

Another aspect of the invention provides a method of modulating receptor function of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to modulate said receptor function.

A further aspect of the invention provides the use of a photodynamic compound for the preparation of a medicament for inhibiting proliferation, inducing apoptosis, inhibiting growth, modulating receptor function, inhibiting signal transduction into the cell or cell signalling and/or inhibiting cellular receptor activation of cells having receptor proteins in combination with illumination of the cells with light in the wavelength interval of 250-305 nm or with light having longer wavelengths that by means of non-linear processes and/or multiphoton excitation promotes the same electronic transitions as light in the wavelength interval of 250-305 nm according to the invention.

A further aspect of the invention provides a method for treatment of a subject, comprising monitoring a subject undergoing a irradiation therapy according to the invention, wherein the monitoring is performed by monitoring with MRI, whether the subject will continue to benefit from the existing irradiation level, and continuing subjecting the subject to radiation therapy if the prediction in the monitoring provides a positive answer.

### SHORT DESCRIPTION OF THE FIGURES

The invention will be further described with reference to the drawings, in which:

FIG. 1 shows the light induced changes in A431 and Cal-39 cells. Laser-pulsed UV illumination blocks EGF receptor signaling in Cal-39 cells. Cal-39 and A431 cells were serum-starved and either treated for 5 minutes with 100 ng/ml EGF prior to or after illumination for 30 minutes. As a control, lysates from serum-starved cells or cells treated with just EGF were loaded. Cell lysates (75 μg) were separated by SDS-PAGE and the membrane probed with the same antibodies described in FIG. 2.

FIG. 2 shows a time series experiment in A431 cells. Laser-pulsed UV illumination of A431 cells blocks EGF receptor signaling. Cells were serum-starved and either treated for 5 minutes with 100 ng/ml EGF prior to or after illumination for the indicated time periods. Cell lysates (75 μg) were separated by SDS-PAGE and the membrane probed with phospho-specific antibodies against the EGF receptor (P-Tyr1173), AKT (P-Thr308) or ERK1/2 (P-Thr202/P-Tyr204). In addition the membranes were probed with antibodies against total EGFR, AKT and ERK1/2 protein.

FIG. 3 shows that illumination of both Cal-39 and A431 cells cause's apoptosis (cell death) as demonstrated by the PARP-cleavage test.

Lane 1: A431 cells illuminated

Lane 2: A431 control cells

Lane 3: Cal-39 cells illuminated

Lane 4: Cal-39 control cells

FIG. 4 shows the 3D structure of the extra- and intracellular domains of the EGFR. Trp residues are grey spheres, Phe and Tyr are white spheres. Disulphide bridges and cysteines are displayed with black sticks. The intracellular domain is a tyrosine kinase (active site Tyr marked by grey arrow).

FIG. 5 shows an overview of the cellular pathways affected by the laser-pulsed UV illumination of the EGF receptor leading to attenuation of the EGFR signaling cascade. The figure shows that UV excitation of the aromatic residues of the EGF receptor will deactivate the EGFR pathways. Without being bound by any theory this deactivation may be due to the fact that the ligand EGF no longer correctly binds the extracellular domain of EGFR due to light induced damage of EGFR receptor 3D structure and/or that laser pulsed UV illumination has damaged the 3D structure of the intra-cellular domain of EGFR or leads to photodegradation of the active site tyrosine residue, that upon activation becomes phosphorylated, again preventing other proteins from binding to the phosphorylated tyrosine residues.

FIG. 6 shows laser-pulsed UV illumination causes an up regulation of p21 irrespective of the p53 status. Cal-39 and A431 cells were serum-starved prior to illumination. Cell lysate (75 μg) was separated by SDS-PAGE and the membrane was probed with a p21^{WAF1}-specific antibody or an antibody specific against p53. To verify equal loading the membrane was also probed with a specific antibody against β-actin.

### DETAILED DESCRIPTION

One aspect of the invention provides a method of inhibiting proliferation of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said proliferation.

Another aspect of the invention provides a method for inducing apoptosis of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to induce said apoptosis.

Another aspect of the invention provides a method of inhibiting growth of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said growth of cells.

Another aspect of the invention provides a method of inhibiting signal transduction into cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said signal transduction into the cell.

Another aspect of the invention provides a method of inhibiting cell signalling of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said cell signalling.

Another aspect of the invention provides a method of inhibiting cellular receptor activation of cells having receptor proteins, said method comprising illuminating the cells with light in the wavelength interval of 250-305 nm or with light having longer wavelengths that by means non-linear processes and/or multiphoton excitation promotes the same electronic transitions as light in the wavelength interval of 250-305 nm to inhibit said cellular receptor activation.

Another aspect of the invention provides a method of modulating receptor function of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to modulate said receptor function.

In one aspect of the invention, the light is in the wavelength interval of 250-300 nm or light having longer wavelengths that by means of non-linear processes and/or multiphoton excitation promotes the same electronic transitions as light in the wavelength interval of 250-300 nm.

In one aspect of the invention, the light is in the wavelength interval of 250-298 nm or light having longer wavelengths that by means of non-linear processes and/or multiphoton excitation promotes the same electronic transitions as light in the wavelength interval of 250-298 nm.

In one aspect of the invention, the method is performed in vivo in a subject.

The present invention provides a method of inhibiting proliferation of cells having receptor proteins in a subject, said method comprising illuminating the cells with light in the wavelength interval of 250-305 nm to inhibit said proliferation.

A further aspect of the invention provides a method for inducing apoptosis of cells having receptor proteins in a subject, said method comprising illuminating the cells with light in the wavelength interval of 250-305 nm to induce said apoptosis.

Another aspect of the invention provides a method of inhibiting growth of cells having receptor proteins in a subject, said method comprising illuminating the cells with light in the wavelength interval of 250-305 nm to inhibit said growth of cells.

Yet a further aspect of the invention provides a method of inhibiting signal transduction into a cell or cell signalling and/or inhibiting cellular receptor activation of cells having receptor proteins, said method comprising illuminating the cells with light in the wavelength interval of 250-305 nm to inhibit said signal transduction into the cell or cell signalling and/or cellular receptor activation.

Yet a further aspect of the invention provides a method of inhibiting signal transduction into cells having receptor proteins in a subject, said method comprising illuminating the cells with light in the wavelength interval of 250-305 nm to inhibit said signal transduction into the cell.

Yet a further aspect of the invention provides a method of inhibiting cell signalling of cells having receptor proteins in a subject, said method comprising illuminating the cells with light in the wavelength interval of 250-305 nm to inhibit said cell signalling.

Yet a further aspect of the invention provides a method of inhibiting cellular receptor activation of cells having receptor proteins in a subject, said method comprising illuminating the cells with light in the wavelength interval of 250-305 nm to inhibit said cellular receptor activation.

In the present context, the term “modulating” refers to either activation or inhibition.

In the present context “inhibiting proliferation” refers to inhibition of cell division of a cell.

In the present context “inducing apoptosis” refers to inducing programmed cell death i.e. that the cell commits suicide.

In the present context “inhibiting growth” refers to inhibition of the size of a cell becoming larger.

In the present context “inhibiting signal transduction in the cell or cell signalling” refers to inhibition of signal transduction downstream of the receptor e.g. by inactivating the receptor so it does not function anymore and thereby does not activate the other signaling molecules in the cascade (downstream).

In the present context “inhibiting cellular receptor activation” refers to inhibition of the receptor such as EGFR. Although the ligand (such as EGF) still may bind to the extra cellular part of the receptor, the receptor's ability to transmit the signal to other molecules is inhibited.

The method according to the invention can be used where inhibition of proliferation, inducing apoptosis, inhibiting growth, inhibiting signal transduction into the cell or cell signalling and/or inhibiting cellular receptor activation of cells having receptor proteins is desired. The method according to the invention can furthermore be used where modulating of receptor function of cells having receptor proteins is desired.

In a further aspect of the invention, the receptor protein is a receptor tyrosine kinase, such as EGFR.

In the present context “receptor tyrosine kinases (RTK's)” refers to RTK's, such as EGF, PDGF, IGF-1, NGF and VEGF receptors, usually present on the surface of a cell. Cells having RTK's can e.g. be identified by using receptor binding studies such as by measuring association kinetics between the ligand (e.g. EGF) and the receptor (e.g. EGFR), where radioactively labelled ligand (e.g. 125I-EGF) is added to the cells to be tested. The cells are washed to remove the excess radioactively labelled ligand. The amount of bound ligand is determined by measuring the radioactivity.

In one aspect of the invention, the cells to be treated are cancer cells. The invention relates in a further aspect to inhibiting cancer cell proliferation. In yet a further aspect, the invention relates to inhibiting cancer cell maturation. In still another aspect, the invention relates to inducing cancer cell apoptosis.

The method according to the invention is particular useful when cells having receptor proteins such as receptor tyrosine kinases have mutated or have an over-expression of receptor tyrosine kinases leading to constitutive signaling and abnormal cell growth. Before treatment a biopsy may be taken in order to verify that the patient's lesions express high amounts of RTK's such as EGFR. This is a routine approach for a pathological laboratory and can e.g. be achieved by applying immuno-histochemistry, where the biopsy material can be fixed with formaldehyde, paraffin-embedded, sliced with a microtome and the EGFR is detected with specific antibodies.

In one aspect of the invention, the cells to be treated have an over-expression of receptor proteins.

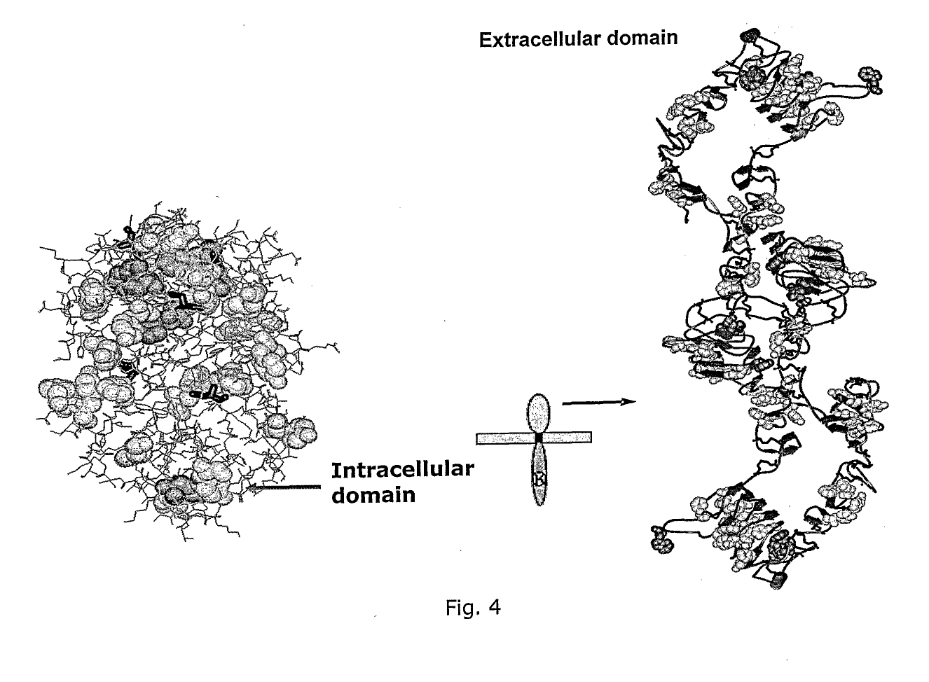

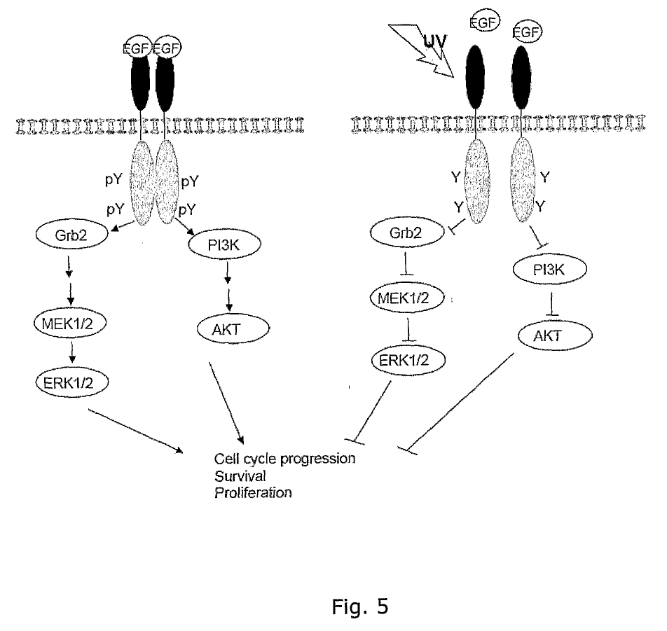

When proteins are exposed to light that excites electronically aromatic residues in proteins such as in the wavelength range from 250 to 305 nm or light with longer wavelengths that by means of non-linear processes and/or multiphoton excitation can promote the same electronic transitions, the light is absorbed by aromatic residues in the proteins. Disulphide bridges are also known to weakly absorb light such as UV light. 3D structure analyses of the kinase membrane receptor EGFR shows that the receptor's extracellular domain is rich in aromatic residues nearby disulphide bridges, and that the intracellular domain is also rich in aromatic residues as seen in FIG. 4.

The spatial proximity between aromatic residues and disulphide bridges in proteins has been preserved during the molecular evolution. Light such as UV illumination of aromatic residues in proteins lead to the disruption of nearby disulphide bridges, leading to free reactive thiol groups, radicals and ions, radicals and ions (Neves-Petersen M T, Klitgaard S, Sundstrom V, Polivka T, Yartsev A, Pasche T, and Petersen S B, in prep.). Interestingly, the EGF receptor is rich in these amino acids known to be involved in photophysical and photochemical reactions triggered by e.g UV light or light that electronically excites the aromatic residues through non-linear processes and/or multiphoton excitation. Without being bound by any theory it is suggested that the absorption of light such as UV light by the receptor proteins in model proteins and aromatic residues can lead to structural changes of these receptors since the excitation of the aromatic residues initiates a cascade of photochemical and photophysical reactions such as electron ejection, radical and ion formation, one of the consequences being disulphide bridge bond breakage as described further below. The close proximity between aromatic residues and disulphide bridges increases the likelihood of SS bond breakage upon illumination with light such as UV light of those nearby aromatic residues. If those aromatic residues are removed from the protein structure, the disruption of the nearby disulphide bridge is minimal or even abolished when the molecule is illuminated with light such as UV light. The 3D structural changes induced by the photochemical and photophysical reactions can cause that the receptor no longer correctly bind its activator and/or that it no longer triggers the downstream phosphorylation reactions leading to e.g. cancer since the internal domain has not been activated. Another possible reason for the light such as UV light induced inhibition of e.g. EGFR is the fact that the active site of the internal tyrosine kinase domain is a tyrosine residue, as shown in FIG. 4, which will be photo-degraded upon UV illumination. As a consequence of photo damage, the active site of the internal domain might putatively no longer be active. Blocking of the downstream reactions leads to inhibition of e.g. uncontrolled cellular proliferation, invasion, metastasis, late-stage disease, chemotherapy resistance, and hormonal therapy resistance. The experimental work done by the present inventors has clearly documented that illumination with light which excites electronically aromatic residues in proteins (such as light in the wavelength interval of 250-305 nm or light with longer wavelengths that by means of non-linear processes and/or multiphoton excitation can promote the same electronic transitions) can inhibit proliferation and induce apoptosis of cells having receptors such as tyrosine kinases, e.g EGFR. The present invention thus presents a new therapy against cancer based on a detailed molecular understanding of UVB effects in proteins including receptor proteins.

As it appears from the examples, the proteins lost their functionality and possibly their native 3D structure above a certain power/illumination time threshold since the transient reactive species formed enter damaging reactions. It is believed that since the receptor proteins targeted with laser UV light treatment such as EGFR are rich in triad of aromatic residues, the mechanism(s) activated by UV light are likely to be partially or fully responsible for induction of cellular inactivation and death, above a certain power/illumination time threshold. Another reason for the UV induced inhibition of EGFR is the fact that the active site of the internal tyrosine kinase domain is a tyrosine residue that will be photo-degraded. Summarising, UV induced structural damages can stop proliferation since the receptor no longer correctly binds its activator and/or no longer triggers the downstream phosphorylative reactions leading to proliferation (such as cancer) since the internal domain has not been activated.

UV light is shown in the examples to prevent that these receptors could be activated and thereby stopping cancer proliferation and it has induced programmed cell death, apoptosis, of cancer cells.

Several reports exist, describing how UV light can activate the EGF receptor hence activating the AKT and MAPK pathway; this is thought to be mediated through (i) increased expression of EGFR ligands and (ii) inactivation of receptor-associated phosphatases and (iii) altered internalization and degradation. These observations are in contrast to our results. Without being bound by any theory the reason for this discrepancy could be found in the illumination power per unit of illuminated area (fluency).

The particular illumination time/fluency threshold to be used when treating a patient will depend on the disease to be treated and on the patient, on the stage of the disease, on a possible combination with other drugs, and on the severity of the case. It is recommended that the light fluency and exposure time be determined for each individual patient by those skilled in the art. In one aspect of the invention, the fluency in the UVB range is above the fluency of light from the sun in the UVB range, known to be of approximately 0.057×10⁻³ kW per m²xnm or 0.002 kW per m².

In one aspect of the invention, a fs laser (femtosecond laser) is used. In this aspect, the energy per pulse is in the interval of 1 picoJ to 30 milliJ, in a further aspect in the interval of 1 nanoJ to 10 milliJ and in yet a further aspect in the interval of 1 nanoJ to 1 milliJ.

In a further aspect of the invention, the laser pulses are from 1 picosecond to 10 millisecond apart, in a further aspect in the interval of 500 picosecond to 1 millisecond apart and in yet a further aspect in the interval of 1 nanosecond to 500 nanosecond apart, and in yet a further aspect in the interval of 10 nanosecond to 200 nanosecond apart.

In a further aspect of the invention, the temporal width of the pulse is in the interval of 1 attosecond to 20 nanosecond, in a further aspect in the interval of 1 femtosecond to 1 nanosecond and in yet a further aspect in the interval 100 femtosecond to 500 femtosecond.

In a further aspect of the invention, the fluency is in the interval of 100 mW/m²xnm to 1000 kW/m² xnm, in a further aspect in the interval of 500 mW/m²xnm to 400 kW/m²xnm, and in yet a further aspect in the interval of 1 kW/m²xnm to 200 kW/m²xnm, and in yet a further aspect in the interval of 10 kW/m²xnm to 100 kW/m²xnm. In one aspect of the invention, the fluency is in the same order of magnitude as used in the experimental section.

It will be apparent to those skilled in the art to change the parameters i.e. energy per pulse, pulse temporal width, and the time between pulses and the equipment used to obtain similar fluencies.

It has been observed that there is an illumination time/power threshold above which the tumor cell death using laser pulsed ultraviolet light can be induced. Below that threshold inhibition of the activation (e.g. phosphorylation) of key proteins involved in the cancer development is not possible. The particular illumination time/power threshold to be used and the optimal intensity to be used when treating a patient will depend on the disease to be treated and on the patient, on a possible combination with other drugs, and on the severity of the case. It is recommended that the irradiation intensity and time be determined for each individual patient by those skilled in the art. The use of drugs that may modulate the target cancer cell sensitivity towards light in conjunction with the light treatment will be beneficial for cancer treatment.

One of the key classes of proteins that have been shown to be targeted using the method according to the invention are RTK's, such as EGFR, especially cells having an overexpression of such receptors. EGFR is e.g. highly expressed in a wide range of solid tumours, many of which are epithelially derived. These include 80-100% of head and neck cancers, 40-80% of non-small-cell lung cancer (NSCLC), 25-77% of colorectal cancers, and 14-91% of breast cancers (Ritter C L, Arteaga C. Semin Oncol 2003; 30 (Suppl 1): 3-11). In one aspect of the invention, the method is useful for treatment of cells that have an over-expression of receptor proteins.

In one aspect of the invention, the cells to be treated are selected from the group consisting of those malignant or non-malignant cells related to surface skin lesions, such as human papillomas, condylomata acuminata, squamous cell carcinomas, vulvar squamous cell carcinoma, vulva condyloma acuminata, vulvar intra-epithelial neoplasm, atrophic type of actinic keratosis, Bowen's disease, mycosis fungoides, erythroplasia of Querat, Gorlin's syndrome, and actinic keratoses; psoriasis, lung cancer and head and neck cancers.

In one aspect of the invention, the cells to be treated are selected form the group consisting of human papillomas, condylomata acuminata, squamous cell carcinomas, vulvar squamous cell carcinoma, vulva condyloma acuminata, vulvar intra-epithelial neoplasm, atrophic type of actinic keratosis, Bowen's disease, mycosis fungoides, erythroplasia of Querat, Gorlin's syndrome, and actinic keratoses.

In one aspect of the invention, the cells to be treated are selected from the group consisting of malignant or non-malignant cells related to surface skin lesions, psoriasis, lung cancer and head and neck cancers.

In one aspect of the invention, the cells to be treated are selected from the group consisting of malignant or non-malignant cells related to surface skin lesions, lung cancer and head and neck cancers.

One aspect of the invention relates to treatment of human patients suffering from skin diseases, i.e. melanoma, non-melanoma skin cancer, inflammatory processes, condyloma acuminata (genital warts), and psoriasis.

In a further aspect of the invention, the cells to be treated are different endogenous cancers such as tumors, which e.g. can be reached by fiber optics, and expressing large amounts of EGFR or mutant EGFR such as e.g. in the case of small cell lung cancer in non-smokers, prostate cancer, esophagus or stomach cancer.

The term “treatment” is defined, as the management and care of a patient for the purpose of combating the disease, condition, or disorder and includes illuminating the patient with light that excites electronically aromatic residues in proteins (such as light in the wavelength interval of 250-305 nm or light with longer wavelengths that by means of non-linear processes and/or multiphoton excitation promotes the same electronic transitions) to prevent the onset of the symptoms or complications, or alleviating the symptoms or complications, or eliminating the disease, condition, or disorder. Treatment includes inhibition of proliferation, inducing apoptosis, inhibiting growth, inhibiting signal transduction into the cell or cell signalling and/or inhibiting cellular receptor activation of cells having receptor proteins such as RTK's e.g. EGFR. In a further aspect of the invention, treatment includes modulating receptor function.

As used herein, the terms “UV light” or “light”, “irradiation” or “UV illumination” or “UV irradiation” or “UV radiation” are a range of wavelengths or a single wavelength of UV light, or IR/visible light for multi-photon excitation.

In one aspect of the invention, the term “light in the wavelength interval of 250-305 nm” refers to a range of wavelengths or a single wavelength of light in the wavelength interval of 250-305 nm, or IR or visible light for multi-photon excitation providing a wavelength in the wavelength interval of 250-305 nm at the place where a therapeutic effect is desired.

In the present context, the term “light that excites electronically aromatic residues in proteins” refers to any light that excites electronically aromatic residues in protein molecules. In one aspect of the invention, the light is in the 250-305 nm range or the energy can be obtained through non-linear processes and/or multiphoton excitation with longer wavelengths that can promote the same electronic transitions.

In the present context “IR” refers to infra red light having a wavelength from 700 nm to 105 nm and visible light refers to light having a wavelength from 700 nm to 400 nm.

In the present context “multi-photon excitation” refers to irradiation performed by multiple-photon excitation, such as two-photon or three-photon irradiation. For example when two-photon excitation is carried out, the sample is irradiated with photons (light) with approximately half the energy (approximately twice the wavelength) of the photons used in a single-photon irradiation. If e.g. a high peak-power, pulsed laser is used (so that the mean power levels are moderate and do not damage the specimen), two-photon events will occur at the point of focus. At this point the photon density is sufficiently high that two photons can be absorbed by simultaneously. This is equivalent to a single photon with energy equal to the sum of the two that are absorbed. In this way, excitation will only occur at the focal point (where it is needed) thereby eliminating excitation of out-of-focus and achieving optical sectioning.

The required irradiation can be performed by e.g. UV light or IR and visible light for multi-photon excitation.

The effect of the treatment according to the invention is without being bound by any theory believed to be the light induced opening of disulphide bridges in the illuminated cells and/or formation of radicals and/or ionic species in the protein due to the light induced processes changing the 3D structure of the proteins rendering them inactive. Although disulphide bridges are commonly found in the structural core and near/on the surface of folded proteins, those located in close proximity to aromatic amino acids are the most susceptible to light induced disruption. During e.g. UV exposure of proteins, energy absorbed by side chains of aromatic amino acid residues is transferred to spatial neighboring disulphide bridges, which function as quenchers (Neves-Petersen M T., et al., 2002, Protein Science 11: 588-600). However, the flow of energy transferred to disulphide bridges and the likely formation of intermediate chemical species such as ejected electrons, radicals/ions formed upon light excitation of the sample ultimately serves to trigger their disruption. The presence of a disulphide bridge with an aromatic residue as a close spatial neighbor in a protein occurs frequently in nature (Petersen M T N., et al., 1999, Protein Engineering 12: 535-548), indicating that photo-induced disulphide bridge disruption is a widespread phenomenon (Petersen M T N., et al., 1999, Protein Engineering 12: 535-548; Neves-Petersen M T et al., 2002, Protein Science 11: 588-600; Vanhooren A et al. 2002, Biochemistry 10; 41(36): 11035-11043).”

As used herein, the term “spatial neighbor” relates to the physical distance between two chemical groups within a composition, such that groups lying in three-dimensional close proximity are considered to be spatial neighbors. A disulphide bridge in e.g. a protein which is a spatial neighbor to an aromatic residue may function as a quencher if the aromatic amino acid absorbs excitation energy following irradiation. The physical distance between half cystines of a disulphide bridge, which are spatial neighbors to one or more aromatic residues such as tryptophan residues and may act as quenchers, can be, but is not limited, to a range up to 15 Å.

Disulphide bridges are known to be excellent quenchers of excited-state aromatic residues. Any aromatic residue, which is in close spatial proximity, can cause photo-induced disruption of a neighbouring disulphide bridge. Hence, the three aromatic amino acids, tryptophan, tyrosine and phenylalanine found in proteins, are all potential mediators of light-induced disulphide bridge disruption. While irradiation with light of a range of wavelengths extending from 250 nm to 305 nm will excite all aromatic residues, the individual aromatic residues have differing absorption maxima (Table 1; data obtained at neutral pH). Absorption and emission maxima will depend on the dielectric constant of the medium (solvent). Table 1 refers to data when the solvent is water.

TABLE 1

| In water | Absorption Max. | Emission Max. |
|---|---|---|
| Phe | 254 nm | 282 nm |
| Tyr | 275 nm | 303 nm |
| Trp | 280 nm | 350 nm |

Since the excitation spectrum of the aromatic amino acid residues is only partially overlapping, protein irradiation at a single narrow wavelength range will excite the individual residues to different degrees. Irradiation at 295 nm can be used to selectively excite tryptophan residues in a protein. Irradiation at 280 nm will excite both tyrosine and tryptophan residues, which can both then cause photo-induced disulphide bridge disruption. Where irradiation is performed by multiple-photon excitation, for example when two-photon excitation is carried out, the sample is irradiated with photons (light) with approximately half the energy (approximately twice the wavelength) of the photons used in a single-photon experiment. For example, electronic excitation of tryptophan can both be achieved with ultraviolet light at 295 nm, or with two-photon excitation at a wavelength of approximately 590 nm. Furthermore, excited tyrosine residues can cause the excitation of neighbouring tryptophan residues by a mechanism called fluorescence resonance energy transfer, which in turn can cause disulphide bridge disruption.

In an aspect of the invention, the irradiation is preferably performed with light having a wavelength of between about 250 nm and about 305 nm. In another aspect of the invention, the wavelength is between about 260 nm and about 300 nm. In yet a further aspect of the invention, the wavelength is between 270 nm and about 295 nm. In yet a further aspect of the invention, the wavelength is between 270 nm and 290 nm. In another aspect of the invention, the wavelength is between 275 nm and about 285 nm. In a further aspect of the invention, the wavelength is about 280 nm.

In an aspect of the invention, the irradiation is preferably performed with light having a wavelength in the interval of 250-305 nm. In an aspect of the invention, the irradiation is preferably performed with light having a wavelength in the interval of 250-300 nm. In another aspect of the invention, the wavelength is in the interval of 260-300 nm. In yet a further aspect of the invention, the wavelength is in the interval of 270-295 nm. In yet a further aspect of the invention, the wavelength is in the interval of 270-290 nm. In another aspect of the invention, the wavelength is in the interval of 275-285 nm. In a further aspect of the invention, the wavelength is about 280 nm.

In another aspect of the invention, the irradiation is performed with light that by means of a non-linear processes and/or multiphoton excitation promotes the same electronic transitions as light with a wavelength of between 250 nm and 305 nm. In another aspect of the invention, the irradiation is performed with light that by means of a non-linear processes and/or multiphoton excitation promotes the same electronic transitions as light with a wavelength of between 250 nm and 300 nm. In another aspect of the invention, the irradiation is performed with light that by means of non-linear processes and/or multiphoton excitation promotes the same electronic transitions as light with a wavelength of between 260 nm and 300 nm. In yet a further aspect of the invention, the irradiation is performed with light that by means of non-linear processes and/or multiphoton excitation promotes the same electronic transitions as light with a wavelength of between 270 nm and 295 nm. In yet a further aspect, the irradiation is performed with light that by means of non-linear processes and/or multiphoton excitation promotes the same electronic transitions as light with a wavelength of between 270 nm and 290 nm. In another aspect of the invention, the irradiation is performed with light that by means of non-linear processes and/or multiphoton excitation promotes the same electronic transitions as light with a wavelength of between 275 nm and 285 nm. In a further aspect of the invention, the irradiation is performed with light that by means of non-linear processes and/or multiphoton excitation promotes the same electronic transitions as light with a wavelength of about 280 nm. In one aspect of the invention, the electronic transistions have been obtained by multiphoton excitation.

In another aspect of the invention, the irradiation step comprises light of a wavelength that specifically excites one or more aromatic amino acids, or other molecular system that may mimic aromatic amino acids, such as light of a wavelength that excites one specific aromatic amino acid such as e.g. the wavelength of approximately e.g. about 295 nm, about 275 nm or about 254 nm that excites respectively tryptophan, tyrosine or phenylalanine, in a further aspect the wavelength at about 295 nm that excites tryptophan or the wavelength about 275 nm that excites tyrosine, or multi-photon excitation, for example 2-photon excitation of between e.g. 500 nm and 640 nm or 3-photon excitation of between 750 nm and 960 nm.

It will be apparent to those skilled in the art that the disruption of disulphide bonds in a given protein at a selected wavelength can be predicted from the location and amino acid neighbours of each disulphide bridge in the 3D structure of the protein. Disulphide bridges placed in the spatial vicinity of aromatic amino acid residues are likely to be the most labile to UV light. The 3D structures of a subset of proteins containing the spatial triad Trp Cys-Cys, in close spatial proximity, have been examined in order to identify which amino acids are located in immediate vicinity of the tryptophan residues of the triad (WO 2004/065928). This analysis has identified those proteins having a similar amino acid neighbourhood composition around the triad to that of cutinase, which can be used to predict which proteins, will have the disulphide bond of the triad broken upon UV illumination.

Light can be applied externally directly onto the patient's skin or internally using optical fibres or in case of metastasis to the patient's blood making use of a dialysis machine.

Light therapy according to the invention can be performed using light with a wavelength of 250-305 nm such as UV light or visible light (e.g. via two-photon excitation) or infra-red light (e.g. via three-photon excitation). In the case of transdermal therapy, the use of infra-red light (via three-photon excitation) would facilitate treatment deeper in the patient. The multi-photon excitation principle allows larger penetration depths since the longer the wavelength of light (e.g. visible and IR light compared to UV light) the deeper light penetrates into a tissue and the less light is scattered by the tissue. Multi-photon excitation offers the additional advantage that excitation only takes place in the focal spot and prevents out of focus bleaching of the fluorescent probe. Optical penetration of tissue may occur down to a depth of 5-7 cm, especially in the case of IR illumination. The main cause for attenuation is scattering events, and therefore tissue density should be taken into account. When IR photons are focused towards a particular target at a depth where scattering events does not lead to severe attenuation of intensity, multi-photon events may be induced, leading to excited states that normally would only occur if visual or UV light was used. The practical depth limits for the successful production of triple photon can be experimentally verified by the skilled person in the art.

When using a multi-photon therapy the beam will be expanded into multiple beams, and using IR transparent optical fibres these beams will be geometrically pointed at the involved tissue, that has been targeted for treatment. The beams will be focused at the target tissue, and will enable multi-photon events. The huge overpopulation of EGFR in some cancer cells, e.g. A431 will be especially susceptible to light induced photo-physical and photochemical processes. The ability to focus will be negatively influenced by the unavoidable scatter of the photon beams by the tissue it passes through. The deepest penetration is achieved with IR—but for superficial targets, visual light can be used. Two photon excitation would promote the same electronic transition that UV light can promote. It is contemplated that far-IR/Microwave sources could be used for the deep tissue illumination and at the focal point promote the same electronic transition that UV light can promote.

In one aspect of the invention, fiber optics are used to deliver the illumination. In another aspect of the invention, the method is applied in treatment of internal organs using laser fiber optics which is endoscopically directed to the cells such as a tumor by illuminating with light in the wavelength interval of 250-305 nm.

A variety of light sources are suitable for the irradiation at a range of wavelengths. As examples mention can be made of lamps e.g. a 75-W Xenon arc lamp from a research grade spectrometer such as a RTC PTI spectrometer, a deuterium lamp, a high pressure mercury lamp. Irradiation at a single wavelength can also be obtained by coupling the light source to a monochromator. As examples of a source of single and multiple photon excitation mention can be made of a high peak-power pulsed or continuous wavelength CW laser.

The source of laser radiation used for the light induced therapy can also be a mode-locked titanium-Sapphire (Ti-Sapphire) laser (Tsunami 3960, Spectra Physics, Mountain View, Calif.) pumped by a high power (5 W at 532 nm) solid state laser (Millennia V, Spectra Physics). The pulsed laser radiation will be sent into a pulse picker that changed its repetition rate to 8 MHz. The 840 nm radiation can be further frequency tripled to 280 nm by using a frequency doubler/tripler (GWU; Spectra Physics).

In one aspect of the invention laser pulsing is used to illuminate the cells according to the method of the invention. In a further aspect continuous wave laser is used to illuminate the cells according to the method of the invention.

In order to follow the light treatment according to the invention, MRI or other methods allowing monitoring possible such as fluorescence spectroscopy, fluorescence lifetime imaging, ultrasound can be used.

The invention thus relates in a further aspect to a method for treatment of a patient, comprising monitoring a patient undergoing light therapy according to the invention, wherein the monitoring is performed
by monitoring with MRI, whether the patient will continue to benefit from the existing irradiation, and
continuing subjecting the patient to irradiation therapy if the prediction in the monitoring provides a positive answer.

Magnetic Resonance Imaging (MRI) is an imaging technique that allows image formation of an arbitrary plane in e.g. a patient. It relies on the use of magnetic properties of atomic nuclei, such as hydrogen. The careful analysis of the radiofrequency absorption and subsequent re-emission in a magnetic field consisting of a constant static field, and a magnetic field gradient that may vary in both time and space enables high quality images with excellent anatomical details, sometimes superimposed with physiological information. Aside from making cross-sectional 3D images, MRI can also be used to generate tissue information in superficial regions such as the skin. Often such studies use so-called surface coils, that improves the sensitivity and thereby the spatial resolution of the local region at the expense of the cross-sectional view. It is anticipated that the UV (or multi-photon) illumination will interfere with normal cell function in the illuminated region. If key functions of the cells are blocked or altered it is highly likely that such cells will have abnormal water content, be that higher or lower. Abnormal water content will result in significant changes in the magnetic relaxation parameters for the tissue in question. By using MRI it is possible to follow the biological effect(s) of light induced chemical changes in the cells. The treatment according to the invention may be correlated with response, and importantly with the penetration depth of the illumination. If the MRI equipment is used to monitor the P-31 NMR signals, a concurrent effect on the high energy phosphates can be mapped as well.

In yet a further aspect of the invention, a method is provided where the irradiation with light according to the invention is combined with use of photodynamic compounds. Conventional photodynamic therapy (PDT) involves a two step protocol which consists of the (selective) uptake and accumulation of a photosensitizing agent in target cells and the subsequent irradiation with light in the visible range. Reactive oxygen species (ROS) produced during this process cause cellular damage and, depending on the treatment dose/severity of damage, lead to either cellular repair/survival, apoptotic cell death or necrosis.

One aspect of the invention thus provides the use of a photodynamic compound for the preparation of a medicament for inhibiting proliferation, inducing apoptosis, inhibiting growth, modulating receptor function, inhibiting signal transduction into the cell or cell signalling and/or inhibiting cellular receptor activation of cells having receptor proteins in combination with illumination of the cells with light in the wavelength interval of 250-305 nm or with light having longer wavelengths that by means of non-linear processes and/or multiphoton excitation promotes the same electronic transitions as light in the wavelength interval of 250-305 nm according to the invention.

The effect of the method according to the invention can be augmented by the use of conventional photodynamic compounds, both mechanisms of actions leading to inhibition of proliferation, inducing apoptosis, inhibiting growth, inhibiting signal transduction into the cell or cell signalling and/or inhibiting cellular receptor activation of cells having receptor proteins.

Examples of photodynamic compounds is Haematoporphyrin and Photofrin; Chlorins and bacteriochlorins; Phthalocyanines; Benzoporphyrin derivative; 5-Aminolaevulinic acid (ALA); purpurins; porphycenes; pheophorbides and verdins.

However, the method according to the invention can also be combined with a photodynamic drug which upon illumination with light in the wavelength interval of 250-305 nm or with visible light is promoted to an electronic excited state that will react with molecular oxygen and promote the formation of reactive oxygen species (ROS), or a drug that have similar excitation spectra as the aromatic residues which when located nearby the cells, e.g. cancer cells could induce the disruption of possible SS bridges or other photo-physical and photo-chemical induced changes such as ion and radical formation in the receptor proteins leading to structural and functional degradation. This treatment involves a two step protocol which consists of the (selective) uptake and accumulation of a photo sensitive drug in target cells and the subsequent irradiation with light in the wavelength interval of specifically 250-305 nm. An example of a photodynamic drug which is activated by light in the wavelength interval of 250-305 nm is BPD verteporfin.

In another aspect of the invention, the method according to the invention can also be combined with a photodynamic drug which upon illumination with visible light is promoted to an electronic excited state that will react with molecular oxygen and promote the formation of reactive oxygen species (ROS). The same visible light could also excite the aromatic residues in proteins due to non-linear processes and/or multiphoton excitation, that would excite electronically the aromatic residues in proteins like light in the wavelength from 250-305 nm does. In this aspect the photodynamic therapy drug would be excited by means of one-photon excitation and the cells would be excited by means of 2-photon excitation.

In another aspect of the invention, the method according to the invention can also be combined with a photodynamic drug which upon illumination with IR light is promoted to an electronic excited state that will react with molecular oxygen and promote the formation of reactive oxygen species (ROS). The same IR light could also excite the aromatic residues in proteins due to non-linear processes and/or multiphoton excitation, that would excite electronically the aromatic residues in proteins like light in the wavelength from 250-305 nm does. In this aspect the photodynamic therapy drug would be excited by means of one-photon excitation and the cells would be excited by means of 3-photon excitation.

When using the method according to the invention a weakening of the targeted tissue might occur and thereby making it more sensitive to chemotherapeutic drugs. In a further aspect, the method according to the invention can be combined with chemotherapeutic drugs. Examples of chemotherapeutic drugs are alkylating agents such as busulfan, cisplatin, carboplatin, chlorambucil, cyclophosphamide, ifosfamide, dacarbazine (DTIC), mechlorethamine (nitrogen mustard), melphalan, and temozolomide; nitrosoureas such as carmustine (BCNU) and lomustine (CCNU); antimetabolites such as 5-fluorouracil, capecitabine, 6-mercaptopurine, methotrexate, gemcitabine, cytarabine (ara-C), fludarabine, and pemetrexed; Anthracyclines and Related Drugs such as daunorubicin, doxorubicin (Adriamycin), epirubicin, idarubicin, and mitoxantrone; topoisomerase I inhibitors such as topotecan and irinotecan: topoisomerase II inhibitors such as etoposide (VP-16) and teniposide; mitotic inhibitors such as the taxanes (such as paclitaxel, docetaxel) and the vinca alkaloids (such as vinblastine, vincristine, and vinorelbine); Corticosteroid Hormones such as prednisone and dexamethasone; L-asparaginase; dactinomycin; thalidomide, and tretinoin.

The method according to the invention can also be combined with the use of drugs or compounds that absorb light in the tissues which are not to be treated (non-targeted tissue) and thereby protect this tissue from radiation damage of the cells there.

Compounds or drugs to be used in combination with the method according to the invention such as photodynamic drugs or chemotherapy drugs can be delivered to the target cells by the use of nanoparticles (such as metal nanoparticles, biological nanoparticles made of, e.g., proteins, or magnetic nanoparticles). Also compounds to protect non-target tissue can be delivered to the non-target cells by the use of nanoparticles (such as metal nanoparticles, biological nanoparticles made of, e.g., proteins, or magnetic nanoparticles).

The method according to the invention can also be combined with the use of nanoparticles such as plasmonic particles which can enhance the intensity of the excitation light e.g if the nanoparticles have a metallic surface such as gold, or have other plasmonic surface.

In yet a further embodiment the use of a photodynamic compound for the preparation of a medicament for inhibiting proliferation, inducing apoptosis, inhibiting growth, inhibiting signal transduction into the cell or cell signalling and/or inhibiting cellular receptor activation of cells having receptor proteins in combination with illumination of the cells with light in the wavelength interval of 250-305 nm, is provided.

In one aspect of the invention, an apparatus for emitting laser pulsed irradiation, the apparatus comprising:
a laser pulsed light provider adapted to emit irradiation which irradiation has a wavelength which excites electronically aromatic residues in proteins,
filtering means adapted to filter irradiation from the irradiation emitter outside the wavelength range exciting electronically aromatic residues in proteins, and
means adapted to guide the filtered radiation onto the surface of a human or a human organ.

In one aspect of the invention, an apparatus is provided for emitting laser pulsed irradiation, the apparatus comprising:
a laser pulsed light provider adapted to emit light which light is in the wavelength interval of 250-305 nm or light having longer wavelengths that by means of a non-linear processes and/or multiphoton excitation promotes the same electronic transitions as light in the wavelength interval of 250-305 nm,
filtering means adapted to filter from the light provider light outside the wavelength range of 250-305 nm or light having longer wavelengths that by means of non-linear processes and/or multiphoton excitation promotes the same electronic transitions as light in the wavelength interval of 250-305 nm, and
means adapted to guide the filtered radiation onto the surface of a human or a human organ.

In another aspect of the invention, an apparatus for emitting laser pulsed irradiation is provided, the apparatus comprising:
a laser pulsed light provider adapted to emit light in the wavelength interval of 250-305 nm,
filtering means adapted to filter light from the light provider outside a wavelength range between 250-305, and
means adapted to guide the filtered radiation onto the surface of a human or a human organ.

In another aspect of the invention an apparatus for emitting light is provided, the apparatus comprising:
a light provider adapted to emit light in the wavelength interval of 250-305 nm or light having longer wavelengths that by means of non-linear processes and/or multiphoton excitation promotes the same electronic transitions as light in the wavelength interval of 250-305 nm,
filtering means adapted to filter from the light provider light outside the wavelength range of 250-305 nm or light having longer wavelengths that by means of non-linear processes and/or multiphoton excitation promotes the same electronic transitions as light in the wavelength interval of 250-305 nm, and
means adapted to guide the filtered radiation onto the surface of a human organ.

In another aspect of the invention an apparatus for emitting irradiation is provided, the apparatus comprising:
a irradiation provider adapted to emit irradiation in the wavelength interval of 250-305 nm,
filtering means adapted to filter irradiation from the irradiation emitter outside the wavelength range between 250-305, and
means adapted to guide the filtered radiation onto the surface of a human organ.

In yet another aspect of the invention an apparatus adapted to emit irradiation is provided, the apparatus comprising:
means for emitting radiation which irradiation has a wavelength which excites electronically aromatic residues in proteins,
a cannula comprising a light guide, and
means for guiding the radiation to the light guide.

In yet another aspect of the invention an apparatus adapted to emit irradiation is provided, the apparatus comprising:
means for emitting radiation in the wavelength range between 250-305,
a cannula comprising a light guide, and
means for guiding the radiation to the light guide.

In yet another aspect, the method according to the invention relates to treatment of cells having receptor proteins performed ex-vivo or in-vitro.

As will be apparent to those of skill in the art upon reading this disclosure, each of the individual embodiments or aspects described and illustrated herein has discrete components and features which may be readily separated from or combined with the features of any of the other several embodiments or aspects without departing from the scope or spirit of the present invention.

### EXAMPLES

In the following we show that LP-UV treatment of two skin-derived tumor cell lines, i.e. A431 and Cal-39 leads to the inhibition of the EGF receptor and key downstream molecules such as AKT1 and ERK1/2 involved in the RTK-catalyzed signaling cascade. These results show a potential for treatment of skin diseases associated with increased proliferation relating to the EGF receptor e.g. warts, condylomas, psoriasis and skin cancer.

### Materials and Methods

### Abbreviations Used:

EGF: epidermal growth factor
AKT: Serine threonine kinase. Also known as protein kinase B.
P-AKT (T308): Phosphorylated AKT (threonine 308, an activating phosphorylation)
ERK1/2: Extracellular signal-regulated kinases 1/2
P-ERK1/2: Phosphorylated extracellular signal-regulated kinases 1/2 again an activating phosphorylation.
EGFR: Epidermal growth factor receptor. A receptor tyrosine kinase.
P-EGFR: Phosphorylated epidermal growth factor receptor, again the phosphorylation is activating.

### Cell Culture

A431 cells (human epidermoid carcinoma cells) were maintained in RPMI medium (Gibco BRL) supplemented with 10% fetal bovine serum (Bio Whittaker Europe). Cal-39 cells (human vulva squamous cell carcinoma cells) were maintained in DMEM medium (Gibco BRL) supplemented with 20% fetal bovine serum (Clonetics), 0.5 nM hydrocortisone and 0.01 μg/ml EGF (Carbiochem.). Both cell lines were obtained from DSMZ, Braunschweig, Germany and kept at 37° C. in a humidified atmosphere and 5% CO₂. For the experiments, 80-85% confluent cells were serum-starved for 18 hours prior to light illumination.

### Illumination Protocol

### Illumination Source

Illumination was carried out with femtosecond lasting laser pulses at a wavelength of 280 nm. The pulses were generated by sending the output from a Spectra physics Tsunami laser (<100 fs pulse duration, 12 nm FWHM, 80 MHz repetition rate, λ840 nm, Tsunami 3960, Spectra Physics, Mountain View, Calif. pumped by a high power (5 W at 532 nm) solid state laser Millennia V, Spectra Physics) through a pulse picker, which decreased the pulse repetition rate to 8 MHz. The fundamental pulse was mixed with its second harmonic (420 nm) in a frequency doubler/tripler (GWU; Spectra Physics) to generate a pulse at 280 nm. The power of the 280 nm light. The power of the 280 nm light after GWU was 0.273 mW. The pulse was expanded prior to sample illumination with a diffusive lens in order to illuminate as large an area as possible (half a petri dish). Each half plate was illuminated at the indicated times. For activation of the EGFR pathway the cells were incubated with 100 ng/ml EGF (Calbiochem) for 5 minutes at 37° C. either prior to or after illumination.

### Illumination Setup During Light Induced Apoptosis Experiments

Both type of cell lines A431 and Cal-39 previously grown in petri dishes non-activated were illuminated for 30 min (each half-plate). Immediately after illumination each plate was incubated for 24 hours at 37° C. Afterwards the cells were harvested.

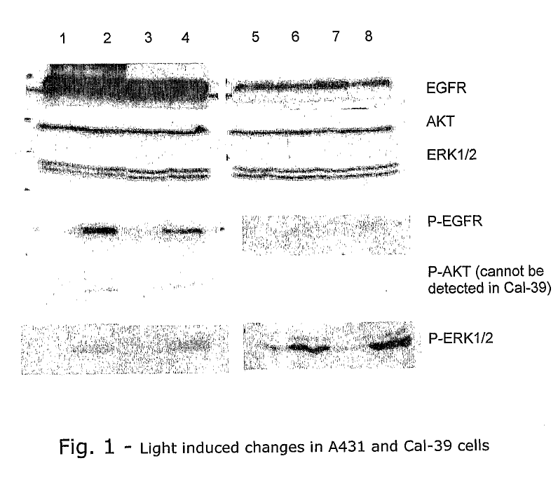

### Illumination Setup Used During the Experiment Reported in FIG. 1

Cell line A431 and Cal49 cultures, previously grown in Petri dishes, were illuminated for 30 min (each half plate) and immediately after illumination EGF (activator) was added for 5 min at 37° C. As one of the control experiments, the cells were activated with EGF (activator) for 5 min at 37° C. and then immediately illuminated for 30 min (each half plate). Afterwards the cells were harvested.

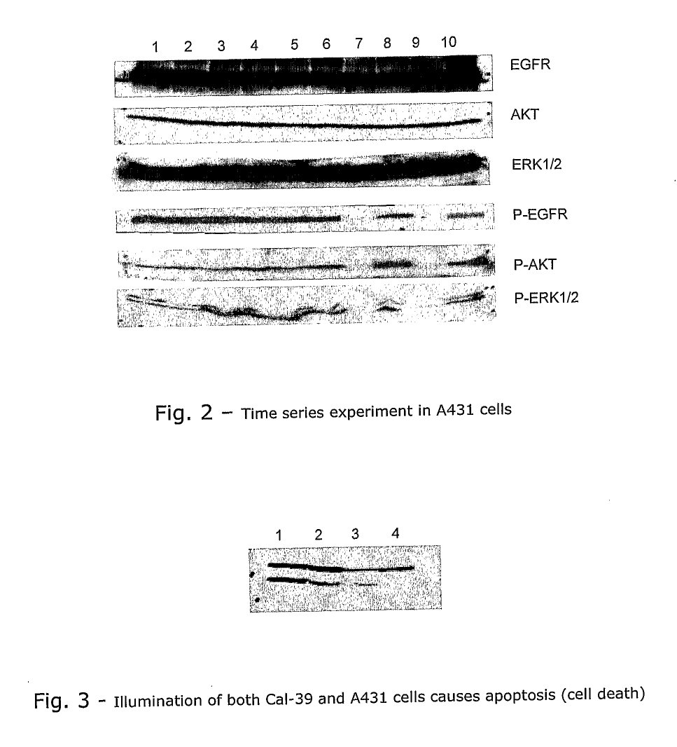

### Illumination Time Series (Results in FIG. 2)

In order to identify the threshold time needed for positive results to occur and to see the result of prolonged illumination time, cell line A431 cultures grown in Petri dishes were illuminated for different amounts of time. Illumination times (per each half plate) were: 15 min, 20 min, 25 min, 30 min and 40 min. Two types of experiments were carried out: a) the cells were illuminated for a particular amount of time prior to adding the EGFR activator. Immediately after illumination EGF (activator) was added for 5 min at 37° C.; b) the cells were activated with EGF (activator) for 5 min at 37° C. and then immediately illuminated for a particular amount of time. Afterwards the cells were harvested.

### EGF Treatment, Western Blotting

Cells were seeded and 48 hours later the cells were serum-starved for 18 hours prior to light immobilization. For activation of the EGFR pathway, cells were either incubated with 100 ng/ml EGF for 5 minutes at 37° C. prior to or after light immobilization.

For detection of apoptosis, the cells were illuminated and the cells further incubated for 24 hours in full medium before they were harvested.

Cells were washed once in icecold PBS and scraped in 50 μl lysis buffer (50 mM Tris-HCl pH 8.5, 150 mM NaCl, 10% Triton X-100, 10% glycerol, 1 mM DTT, 30 mM NaPP_{i, 10} mM NaF, 1 mM Na₃VO₄, 100 nM okadaic acid, Complete protease inhibitors (Roche)). Lysates were incubated on ice for 15 minutes and cleared by centrifugation (4° C., 12000×g, 30 min). The supernantant was used for protein determination according to Bradford (1976).

75 μg of protein was separated by 10% SDS-PAGE and the proteins transferred to a PVDF membrane (Biorad) by wetblotting in 25 mM Tris, 192 mM glycine, 0.1% SDS, 20% MeOH. The membrane was blocked in blocking buffer (0.2% casein, 0.1% Tween20 in PBS) for 1 hour and incubated with monoclonal anti-AKT (BD Transduction Laboratories), anti-PARP (Pharmingen), anti-β-actin (Sigma) or polyclonal anti-Phospho-AKT (T308) (Cell Signaling), Anti-ERK1/2 (p42/p44) (Cell Signaling), Anti-Phospho-ERK1/2 (P-p42/p44) (Thr202/Tyr204) (Cell Signaling), Anti-EGFR (Santa Cruz), Anti-Phospho-EGFR (Tyr1173) (Santa Cruz).

After washing in blocking buffer the membranes were incubated with either a secondary goat-anti-mouse antibody or goat-anti-rabbit or sheep-anti-goat coupled to alkaline phosphatase (Jackson immunoresearch laboratories) for 1 hour. Visualization was done using CDP-star (Tropix) according to the manufacturer's instructions.

### Results

### Experiment 1

### Illumination of Two Types of Carcinogenic Cells Demonstrating that UV Illumination Prior to Activation of the EGFR Leads to Inactivation of EGFR and to Changes of Metabolic Pathways (Since Phosphorylated Form of Downstream Proteins are No Longer Seen)

To see if the observed blockage of EGFR signaling in A431 cells (see below experiment 2) also is found in other cell lines, illumination experiments were performed in another human skin cancer cell line, i.e. Cal-39, which expresses lower levels of the EGF receptor (compare FIGS. 1A and 1B). Western blot detection of phosphorylated EGFR, AKT1 and ERK1/2 was used to assess the effect of the UV illumination.

FIG. 1A shows the results from the human squamous cell line A431. The cells were serum-starved prior to incubation with EGF and illumination. As expected, serum-starved cells (lane 1; control) show no phosphorylation of either EGFR, AKT1 or ERK1/2, whereas incubation of cells with EGF lead to activation of the EGFR signaling cascade assessed by phosphorylation of both the EGF receptor, and the two downstream effectors AKT and ERK1/2 (Lane 2; EGF). EGF incubation prior to illumination did not change its ability to activate the phosphorylation cascade (lane 4; EGF, UV), whereas UV illumination prior to EGF treatment prevented activation of the EGFR, AKT1 and ERK1/2 (Lane 4; UV, EGF), hence there is no effect of EGF once the cells have been illuminated. Western blot detection of total EGFR, AKT and ERK1/2 proteins show that the protein level is not affected by illumination.

The results using Cal-39 cells were the same (FIG. 1B) as for A431 cells. Illumination prior to EGF incubation prevented activation of the EGFR signaling cascade, whereas illumination after incubation with EGF had no effect on the activation of the signaling pathway. These results support the hypothesis that laser-pulsed UV can impact the EGF receptor and prevents its activation by ligand binding.

FIG. 1 shows the light induced changes in A431 and Cal-39 cells.

Lane 1: A431: Illumination 30 min, then EGF

Lane 2: A431: EGF, then illumination 30 min

Lane 3: A431: Control (no light, no EGF)

Lane 4: A431: EGF (no light)

Lane 5: Cal-39: Illumination 30 min, then EGF

Lane 6: Cal-39: EGF then illumination 30 min

Lane 7: Cal-39: Control (no light, no EGF)

Lane 8: Cal-39: EGF (no light)

The upper half of the figure is a control showing the protein content, whereas in the lower half only the phosphorylated forms of the proteins are detected.

Lane 3 and lane 7 are controls showing that the serum starvation has worked ant the EGF pathway is not active. Lane 4 and 8 are also controls showing that upon addition of EGF, the pathway becomes activated as seen by the phosphorylation of three components in the EGF signaling pathway.

Lane 2 and 6 show that EGF treatment prior to illumination does not prevent activation of this pathway, whereas EGF treatment after illumination shows no activation of the pathway (lane 1 and 5).

This means that illumination can block the effect of EGF and the signaling transduction cascade involving the upstream EGF receptor is halted when the cells are illuminated.

### Experiment 2

### With the Cell Line A431, an Illumination Time Series were Done in Order to Identify the Threshold Time Needed for Positive Results to Occur and to See the Result of Prolonged Illumination

The human skin cancer cell line, A431, which overexpress the EGF receptor (more than 1.5 million receptors per cell) was used to investigate whether laser-pulsed UV illumination could block EGF receptor signaling. Interestingly, the EGF receptor contains aromatic residues in close proximity to S—S bridges making it a likely candidate for light-induced immobilization.

To assess the time required to inactivate the EGF receptor with UV light, a time course experiment was performed in A431 cells (FIG. 2). Cells were serum-starved prior to treatment and the cells were either illuminated at different time points and then incubated with EGF or first treated with EGF followed by different UV light illumination times. The blockage in EGF receptor signaling was detected by western blotting using phosphospecific antibodies against the EGFR receptor and the downstream signaling molecules AKT1 and ERK1/2. As seen in FIG. 2, there exists a power/threshold level above which it is possible to inactivate the EGF receptor, as seen by no phosphorylation of the EGF receptor and the downstream signaling molecules AKT and ERK1 and 2 (Lanes 7-10), whereas below this threshold (Lanes 1-6) no inactivation could be detected by western blotting. Detection of total EGFR, AKT1 and ERK1/2 protein was used to ensure equal loading.

FIG. 2 shows time series experiment in A431 cells

Lane 1: Illumination 15 min, then EGF

Lane 2: EGF then illumination 15 min

Lane 3: Illumination 20 min, then EGF

Lane 4: EGF, then illumination 20 min

Lane 5: Illumination 25 min, then EGF

Lane 6: EGF, then illumination 25 min

Lane 7: Illumination 30 min, then EGF

Lane 8: EGF, then illumination 30 min

Lane 9: Illumination 40 min, then EGF

Lane 10: EGF, then illumination 40 min

The proteins detected are the same as in FIG. 1.

This figure shows that there is a certain threshold above which illumination halts the signaling pathway. The results show that illumination times for more than 30 minutes attenuate the EGFR signaling pathways.

### Experiment 3

### Light Induced Apoptosis Experiment

FIG. 3 shows the illumination of both Cal-39 and A431 cells causes apoptosis (cell death).

Lane 1: A431 cells illuminated

Lane 2: A431 control cells

Lane 3: Cal-39 cells illuminated

Lane 4: Cal-39 control cells

The upper band represents full length PARP and the lower band the cleaved form. As can be seen the intensity of the lower band increases when the cells are illuminated, indicating the cells are apoptotic. This is seen for both cell lines.

### Experiment 4

### Laser-Pulsed UV Illumination Upregulates p21^{WAF1} Irrespective of the p53 Status

Treatment of cells with monoclonal antibodies against the EGF receptor or tyrosine kinase inhibitors have been shown to up regulate the cyclin dependent kinase inhibitor, p21^{WAF1} (36-40).

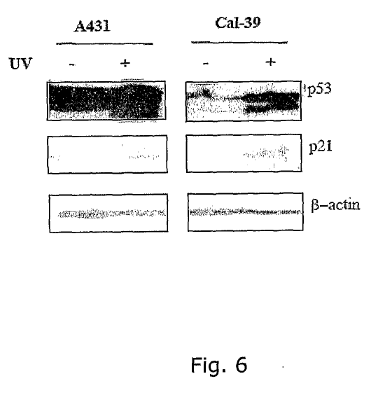

To test if p21^{WAF1} was up regulated in response to laser-pulsed UV illumination, both serum-starved Cal-39 and A431 cells were illuminated and returned to full medium and incubated for 24 hours. The levels of p21^{WAF1} in illuminated cells in response to not illuminated cells were detected by western blotting using an anti-p21^{WAF1} specific antibody. The western blot (FIG. 6) shows that p21 is indeed up regulated in response to UV illumination. p21^{WAF1} is a p53 responsive gene, hence we tested the expression of p53 in response to illumination. The A431 cells express mutated p53, which is not up regulated in response to illumination, whereas Cal-39 cells express wild type p53, which is up regulated in response to illumination (FIG. 6). Although p53 is mutated in A431 cells, both cell lines show an up regulation of p21^{WAF1} in response to illumination.

### REFERENCES

Bradford M M. (1976). A rapid and sensitive method for the quantitation of microgram quantities of protein utilizing the principle of protein-dye binding. Anal Biochem. May 7; 72:248-54.

1. A method of inhibiting proliferation or growth of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said proliferation or growth of cells.

2. A method for inducing apoptosis of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to induce said apoptosis.

3. (canceled)

4. A method of inhibiting cell signaling or signal transduction into cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said cell signaling or signal transduction into the cell.

5. (canceled)

6. A method of inhibiting cellular receptor activation or modulating receptor functions of cells having receptor proteins, said method comprising illuminating the cells with light in the wavelength interval of 250-305 nm or with light having longer wavelengths that by means of non-linear processes and/or multiphoton excitation promotes the same electronic transitions as light in the wavelength interval of 250-305 nm to inhibit said cellular receptor activation or modulation of cellular receptor functions.

7. (canceled)

8. The method according to any one of claims 1, 2, 4 or 6, wherein the light has a wavelength in the interval of 250 nm-300 nm.

9. The method according to any one of claims 1, 2, 4, or 6, wherein the method is performed in vivo in a subject.

10. The method according to any one of claims 1, 2, 4, or 6, wherein said electronic transitions have been obtained by multiphoton excitation.

11. The method according to any one of claims 1, 2, 4, or 6, wherein the receptor protein is a receptor tyrosine kinase.

12. The method according to any one of claims 1, 2, 4, or 6, wherein the receptor tyrosine kinase is EGFR.

13. The method according to any one of claims 1, 2, 4, or 6, wherein the cell is a cancer cell.

14. The method according to any one of claims 1, 2, 4, or 6, wherein the cells to be treated are selected from the group consisting of malignant or non-malignant cells related to surface skin lesions, psoriasis, lung cancer and head and neck cancers.

15. The method according to any one of claims 1, 2, 4, or 6, wherein the cells are selected form the group consisting of human papillomas, condylomata acuminata, squamous cell carcinomas, vulvar squamous cell carcinoma, vulva condyloma acuminata, vulvar intra-epithelial neoplasm, atrophic type of actinic keratosis, Bowen's disease, mycosis fungoides, erythroplasia of Querat, Gorlin's syndrome, and actinic keratoses.

16. The method according to any one of claims 1, 2, 4, or 6, wherein the cells have an over-expression of receptor proteins.

17. The method according to any one of claims 1, 2, 4, or 6, wherein the light has a wavelength in the interval of 260-300 nm.

18. The method according to any one of claims 1, 2, 4, or 6, wherein the light has a wavelength in the interval of interval of 270-295 nm.

19. The method according to any one of claims 1, 2, 4, or 6, wherein the light has a wavelength in the interval of 275-285 nm.

20. The method according to any one of claims 1, 2, 4, or 6, wherein fiber optics is used to illuminate the cells.

21. The method according to any one of claims 1, 2, 4, or 6, wherein pulsed laser radiation is used to illuminate the cells.

22. The method according to any one of claims 1, 2, 4, or 6, wherein fiber optics are used for endoscopically illuminating the cells of internal organs.

23. The method according to any one of claims 1, 2, 4, or 6, combined with the use of photodynamic compounds.

24. The method according to any one of claims 1, 2, 4, or 6, combined with the use of chemotherapeutic drugs.

25. The method according to claim 23 combined with the use of nanoparticles for delivering the compounds to the target cells.

26. The method according to claim 25, wherein the nanoparticles are plasmonic particles.

27. The method according to any one of claims 1, 2, 4, or 6, combined with the use of compounds that protect the non-target tissue from radiation damage.

28. The method according to claim 27 combined with the use of nanoparticles for delivering the compounds to the non-target cells.

29. The method according to claim 28, wherein the nanoparticles are plasmonic particles.

30. A method of preparing a medicament, said method comprising a photodynamic compound in combination with at least one of: illumination of cells with light in the wavelength interval of 250-305 nm or with light having longer wavelengths that by means of non-linear processes or multiphoton excitation promotes the same electronic transitions as light in the wavelength interval of 250-305 nm wherein said medicament has properties comprising at least one of: inhibiting proliferation, inducing apoptosis, inhibiting growth, modulating receptor function, inhibiting signal transduction into a cell or cell signalling and/or inhibiting cellular receptor activation of cells having receptor proteins.

31. A method for treatment of a subject, comprising monitoring a subject undergoing a irradiation therapy according to any one of claims 1, 2, 4, or 6, wherein the monitoring is performed by monitoring with MRI, whether the subject will continue to benefit from the existing irradiation level, and continuing subjecting the subject to radiation therapy if the prediction in the monitoring provides a positive answer.

32. The method according to claim 31, wherein the treatment of cells having receptor proteins are performed ex-vivo.

33. The method according to claim 31, wherein the treatment of cells having receptor proteins are performed in-vitro.

34. An apparatus for emitting laser pulsed irradiation, the apparatus comprising:
a laser pulsed light provider adapted to emit irradiation which irradiation has a wavelength which excites electronically aromatic residues in proteins,
filtering means adapted to filter irradiation from the irradiation emitter outside the wavelength range exciting electronically aromatic residues in proteins, and
means adapted to guide the filtered radiation onto the surface of a human or a human organ.

35. The method according to claim 24 combined with the use of nanoparticles for delivering the compounds to the target cells.

36. The method according to claim 35, wherein the nanoparticles are plasmonic particles.
